(19) United States
(12) Patent Application Publication
(10) Pub. No.: US 2026/0248517 A1
(43) Pub. Date: Aug. 27, 2026

(54) MEDICAL DRILL AND IMPLANT DEVICE AND METHODS OF USING THE SAME

(71) Applicant: Peninsula Surgical Solutions, LLC, San Pedro, CA (US)

(72) Inventors: Allan DELMAN, Rolling Hills, CA (US); Connor DELMAN, Westlake Village, CA (US); Quinten KUNZ, Richmond, UT (US); Geoffrey WHITE, Hyde Park, UT (US); Bruce HOFFMAN, Smithfield, UT (US); Tyler PULSIPHER, Logan, UT (US)

(73) Assignee: Peninsula Surgical Solutions, LLC, San Pedro, CA (US)

(21) Appl. No.: 19/550,799

(22) Filed: Feb. 26, 2026

Related U.S. Application Data
(60) Provisional application No. 63/796,509, filed on Apr. 29, 2025.
(60) Provisional application No. 63/764,389, filed on Feb. 27, 2025.

(51) Int. Cl. A61B 17/16; A61B 90/00; A61B 17/00

(52) U.S. Cl. CPC A61B 17/1626; A61B 17/162; A61B 17/1628; A61B 90/06; A61B 2017/00022; A61B 2017/00398; A61B 2017/00477; A61B 2017/0092; A61B 2090/066

(57) ABSTRACT

The present disclosure is directed to a drill and implant device and methods for using the same. The device is configured to automatically drill into, for example, a bone to different depths depending on whether the device is in a manual mode, a first cortex drill mode, a second cortex find mode, a second cortex drill mode, a fixed depth mode, or a lag screw mode.

### BACKGROUND

### Technical Field

The present disclosure is directed to a medical device and methods for using the same.

### Description of the Related Art

Orthopedic surgeons treat musculoskeletal system ailments, such as injuries to bones, joints, and ligaments. This may require the insertion of a fastener or implant, such as a screw or pin, into bone. Generally, the implant is inserted into the bone by drilling a hole into the bone with a drill bit, removing the drill bit from the hole, measuring the depth of the hole with a depth gauge, inserting the implant into the hole with a screwdriver, and repeating this process for each implant. This process has several drawbacks.

One drawback is that the orthopedic surgeon uses physical force to push the drill while driving the drill bit into the bone. Similarly, manual force is used to withdraw the drill bit and to insert the screw into the hole with a screwdriver. These actions, which are often done repetitively, are fatiguing.

Furthermore, drilling a hole in bone often requires substantial force and may result in the inadvertent passage of the drill too far, potentially injuring the patient by penetrating the soft tissue. In addition, the surgeon may mistakenly insert the screw into the hole at the wrong angle or trajectory impairing the strength or effectiveness of the implant.

In addition, current drills do not detect when a bone cortex has been penetrated or the length between a near and a far bone cortex. Instead, depth gauges are typically used. Unfortunately, it is often difficult for the surgeon to obtain an accurate measurement of depth using the depth gauge because it requires tactile feedback, which can be demanding in the clinical setting. If the depth measurement is inaccurate, the surgeon may insert a screw of the wrong length, which should then be removed and discarded, resulting in wasted hardware and increased costs. In addition, if the depth measurement is inaccurate or technically difficult, verification may require repeated radiographs, which is time consuming. As a result, patients are subjected to longer anesthesia times and there is greater radiation exposure to the patient, the surgeon, and the ancillary medical staff.

### BRIEF SUMMARY

The present disclosure is directed to a drill and implant device and methods for using the same.

The device is configured to automatically drill a hole into an object, such as a bone, stop once a desired depth has been reached, retract automatically, and subsequently insert an implant into the hole. The device drills the hole and inserts the implant without physical intervention on part of a user. More specifically, in contrast to traditional drills and screwdrivers, the user does not apply a physical force to the device to drill the hole or insert the implant. Rather, the device drills the hole and inserts the implant under its own power. In addition, the user does not move the device when switching between drilling the hole to inserting the implant. Instead, the device may remain stationary during both the drilling of the hole and the insertion of the implant. Accordingly, surgical efficiency and accuracy are improved, and radiation exposure is reduced.

The device is configured to operate in a plurality of different operation modes including a manual mode, a first cortex drill mode, a second cortex find mode, a second cortex drill mode, a fixed depth mode, and a lag screw mode. The device automatically drills into a target bone to different depths depending on the operation mode. Other modes are also possible.

### BRIEF DESCRIPTION OF THE SEVERAL VIEWS OF THE DRAWINGS

In the drawings, identical reference numbers identify similar features or elements. The size and relative positions of features in the drawings are not necessarily drawn to scale.

FIG. 1 is a first angled view of a device according to an embodiment disclosed herein.

FIG. 2 is a second angled view of a device according to an embodiment disclosed herein.

FIG. 3 is a first internal view of a device according to an embodiment disclosed herein.

FIG. 4 is an exploded view of a device according to an embodiment disclosed herein.

FIG. 5 is a second internal view of a device according to an embodiment disclosed herein.

FIG. 6 shows a loading of a drill bit into a device according to an embodiment disclosed herein.

FIG. 7 shows a target bone according to an embodiment disclosed herein.

FIG. 8 is a block diagram of a method of operating a manual mode of a device according to an embodiment disclosed herein.

FIG. 9 is a block diagram of a method of operating a first cortex drill mode of a device according to an embodiment disclosed herein.

FIG. 10 is a block diagram of a method of operating a second cortex find mode of a device according to an embodiment disclosed herein.

FIG. 11 is a block diagram of a method of operating a second cortex drill mode of a device according to an embodiment disclosed herein.

FIG. 12 is a block diagram of a method of operating a fixed depth mode of a device according to an embodiment disclosed herein.

FIG. 13 is a block diagram of a method of operating a lag screw mode of a device according to an embodiment disclosed herein.

FIG. 14 is a radiolucent attachment according to an embodiment disclosed herein.

### DETAILED DESCRIPTION

In the following description, certain specific details are set forth in order to provide a thorough understanding of various aspects of the disclosed subject matter. However, the disclosed subject matter may be practiced without these specific details. In some instances, well-known structures and methods of using electrical components and devices, such as drills, screwdrivers, and sensors, have not been described in detail to avoid obscuring the descriptions of other aspects of the present disclosure.

Unless the context requires otherwise, throughout the specification and claims that follow, the word “comprise” and variations thereof, such as “comprises” and “comprising,” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present disclosure.

The present disclosure is directed to a drill and implant device 10 and methods for using the same.

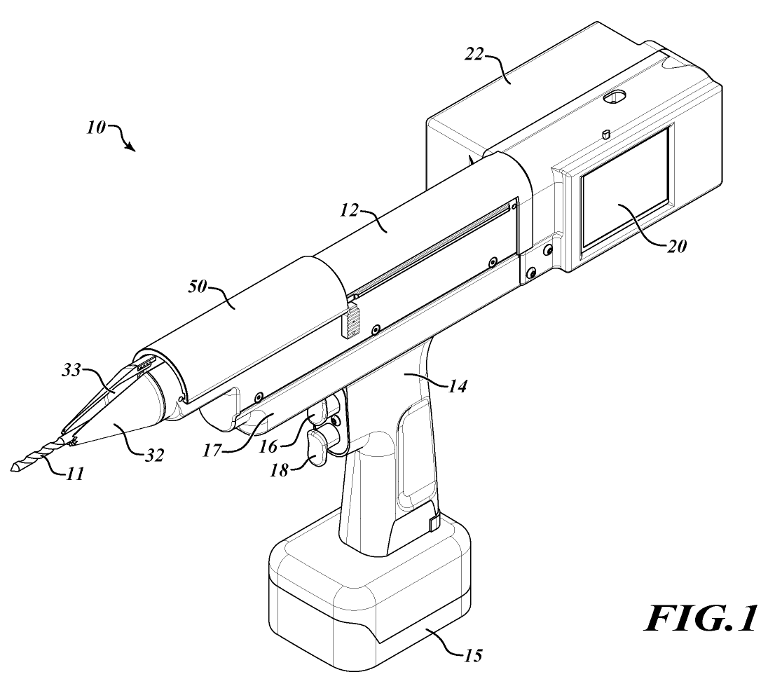

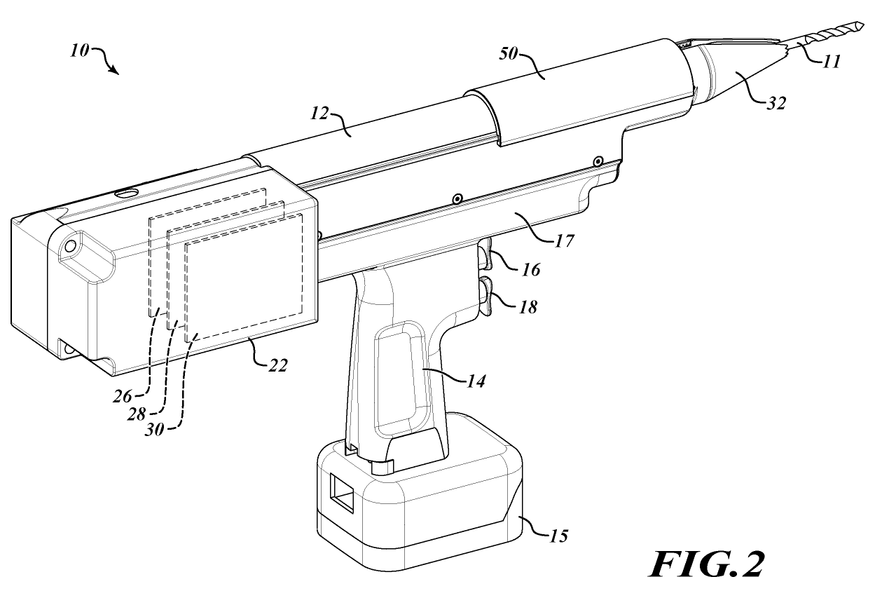

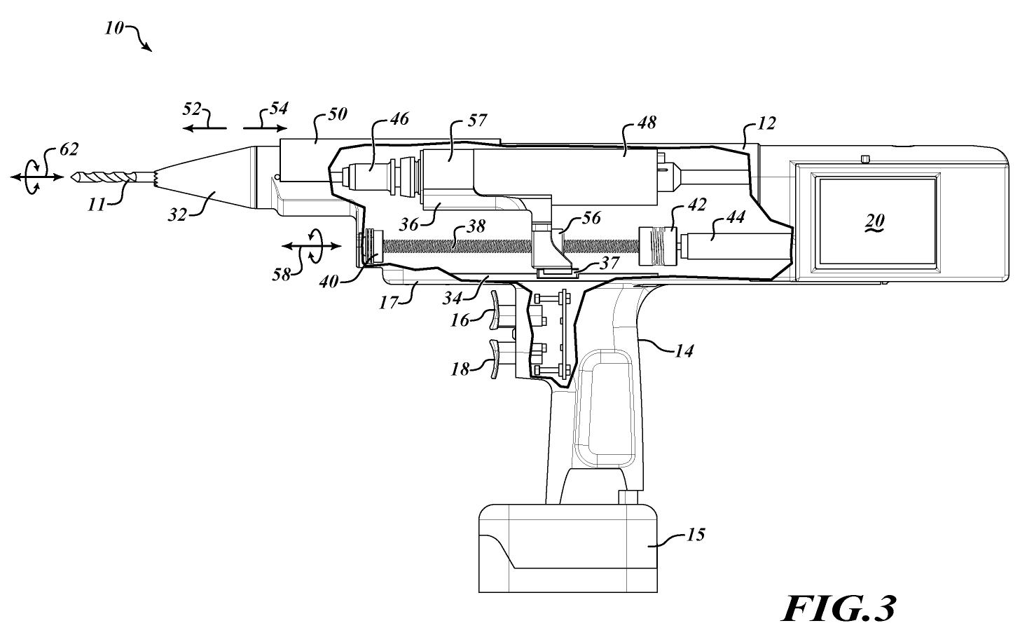

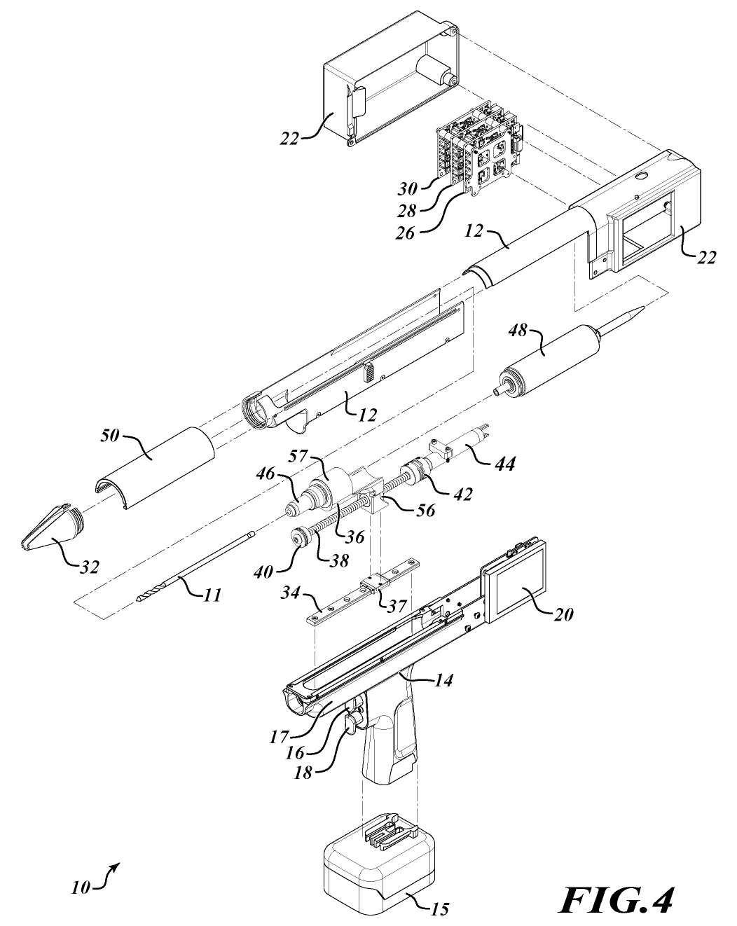

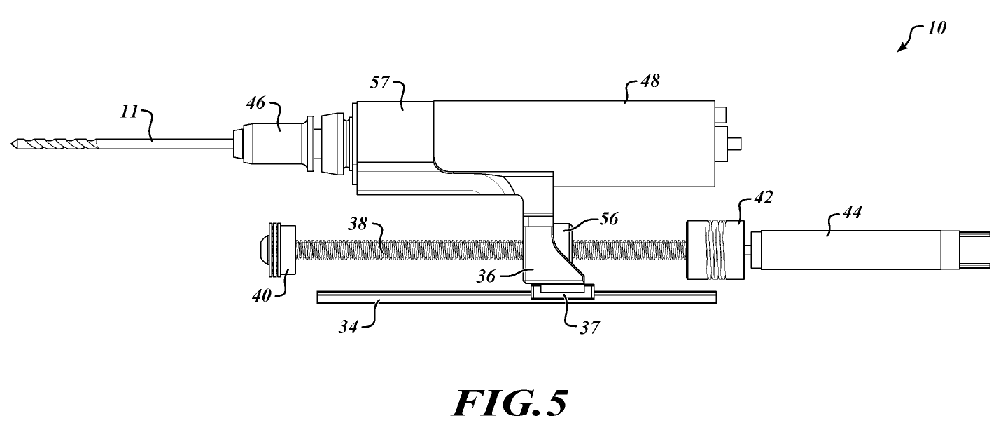

FIG. 1 is a first angled view of the device 10 according to an embodiment disclosed herein. FIG. 2 is a second angled view of the device 10 according to an embodiment disclosed herein. FIG. 3 is a first internal view of the device 10 according to an embodiment disclosed herein. FIG. 4 is an exploded view of the device 10 according to an embodiment disclosed herein. FIG. 5 is a second internal view of the device 10 according to an embodiment disclosed herein. It is beneficial to review FIGS. 1, 2, 3, 4, and 5 together.

The housing of the device 10 is removed in FIG. 5. Further, the device 10 is mounted with a drill bit 11 in FIGS. 1-5. However, as will be discussed in further detail below, the device 10 may be mounted with various other tools, such as drill bits, screws, pins, reamers, and anchors.

The device 10 includes a housing 12, a handle 14, a battery 15, an upper trigger 16, a lower trigger 18, a user interface 20, a controller cover 22, a linear controller 26, a drill controller 28, an application controller 30, a nose cone 32, a rail 34, a carriage 36, a lead screw 38, a bearing structure 40, a shaft coupling 42, a linear motor 44, a chuck 46, and a drill motor 48.

The housing 12 is a protective body that contains internal components of the device 10. The internal components will be discussed in further detail below. The housing 12 includes a door 50 that opens to expose the internal components of the device 10, as well as allow for tools to be loaded into the device 10. The loading of tools will be discussed in further detail below.

The handle 14 is coupled to the housing 12. The handle 14 allows a user to hold and handle the device 10 with a single hand or with two hands. The handle 14 may also be placed and secured in a holder for remote operation. The handle 14, in addition to the housing 12, may contain internal components of the device 10, such as a processor and various electrical components.

The handle 14 includes a support portion 17 that provides a platform for the other components of the device 10. For example, the user interface 20, the controller cover 22, the linear controller 26, the drill controller 28, the application controller 30, the nose cone 32, the rail 34, the carriage 36, the lead screw 38, the bearing structure 40, the shaft coupling 42, the linear motor 44, a chuck 46, and the drill motor 48 are positioned on the support portion 17.

The battery 15 is coupled to the handle 14. The battery 15 is a power source that provides electrical power for the various components of the device 10. The battery 15 also provides a base or support for the device 10. The battery 15 may be any type of power source that provides electrical power.

The upper trigger 16 and the lower trigger 18 are positioned on the handle 14. The functions of the upper trigger 16 and the lower trigger 18 change depending on the current settings of the device 10 and operation mode of the device 10. For example, in case the upper trigger 16 and the lower trigger 18 are set to control the linear motor 44, the upper trigger 16 operates the linear motor 44 to linearly move the drill bit 11 in a forward direction 52 (referring to FIG. 3) out of the nose cone 32, and the lower trigger 18 operates the linear motor 44 to linearly move the drill bit 11 in a backward direction 54 (referring to FIG. 3) into the nose cone 32. As another example, in case the upper trigger 16 and the lower trigger 18 are set to control the drill motor 48, the upper trigger 16 operates the drill motor 48 to rotate the drill bit 11 in a clockwise direction, and the lower trigger 18 operates the drill motor 48 to rotate the drill bit 11 in counter-clockwise direction (or vice versa). As another example, one of the upper trigger 16 or the lower trigger 18 initiates an operation mode of the device 10 and the other one of the upper trigger 16 or the lower trigger 18 stops the operation mode of the device 10. Operation modes of the device 10 will be discussed in further detail below. The upper trigger 16 and the lower trigger 18 are also coupled to a sensor, such as a Hall Effect sensor, to measure how much each of the upper trigger 16 and the lower trigger 18 have been pulled (e.g., how much pressure is applied). The speed of, for example, the linear motor 44 or the drill motor 48 is adjusted proportionally to how much the upper trigger 16 and the lower trigger 18 have been pulled.

The user interface 20 is positioned on the support portion 17 in the housing 12. The user interface 20 is positioned on an opposite side of the device 10 from the nose cone 32. The user interface 20 may be positioned at other locations, such as on the handle 14. The user interface 20 may also be located at a remote location from the device 10, and communicate with remaining components of the device 10 wirelessly though, for example, Wi-Fi and Bluetooth. The user interface 20 may be any type of user interface, such as a touch display and a display with a plurality of user inputs.

The user interface 20 displays information to a user, as well as receives input from the user. The information includes various types of data and parameters related to the device 10 and processes performed by the device 10. For example, the user interface 20 displays a size and type of tool (e.g., drill bit, screwdriver bit) and implant (e.g., screw or pin) currently loaded in the device 10, measurements generated by the device 10 (e.g., depth measurement of a current hole being drilled), and parameters of the device 10 (e.g., current torque level, current drilling or screwing speed, current power level). The information also includes various operation modes of the device 10 that may be selected by the user through the user interface 20 and status information of the various operation modes (e.g., the current total time of performing a particular operation mode). The different operation modes of the device 10 will be discussed in further detail below.

The controller cover 22 is positioned on the support portion 17 in the housing 12. The controller cover 22 provides a housing or enclosure for the linear controller 26, the drill controller 28, and the application controller 30. The controller cover 22 is positioned on an opposite side of the device 10 from the user interface 20.

The linear controller 26 is positioned in the controller cover 22. The linear controller 26 is communicatively coupled to the linear motor 44 and controls operation of the linear motor 44. For example, the linear controller 26 instructs the linear motor 44 to rotate the lead screw 38 in a first direction such that the drill bit 11 linearly moves in the forward direction 52 out of the nose cone 32, and instructs the linear motor 44 to rotate the lead screw 38 in a second, opposite direction such that the drill bit 11 linearly moves in the backward direction 54 into the nose cone 32. The linear controller 26 also monitors various parameters, such as revolutions per minute (RPM) or revolutions per second (RPS), operation time, and operation history, of the linear motor 44.

The drill controller 28 is positioned in the controller cover 22. The drill controller 28 is communicatively coupled to the drill motor 48 and controls operation of the drill motor 48. For example, the drill controller 28 instructs the drill motor 48 to rotate the drill bit 11 in a clockwise direction, and instructs the drill motor 48 to rotate the drill bit 11 in counter-clockwise direction. The drill controller 28 also monitors various parameters, such as RPM or RPS, operation time, and operation history, of the drill motor 48.

The application controller 30 is positioned in the controller cover 22. The application controller 30 is communicatively coupled to the linear controller 26 and the drill controller 28. The application controller 30 executes various programs or applications, and provides corresponding instructions to the linear controller 26 and the drill controller 28, which in turn, provide instructions to the linear motor 44 and the drill motor 48, respectively. For example, as will be discussed in further detail below, the application controller 30 controls the device 10 to perform various operation modes.

The linear controller 26, the drill controller 28, and the application controller 30 may each be any type of controller, processor, or application specific integrated circuit (ASIC) that executes instructions stored in a memory of the device 10.

The nose cone 32 is coupled to a front end of the housing 12. The nose cone 32 is aligned with the chuck 46 and the drill motor 48. The nose cone 32 includes a guide portion 33 (referring to FIG. 1) that provides a guide for the tool loaded into the chuck 46 and exiting out of the nose cone 32. In operation, the nose cone 32 is placed at a target location (e.g., femur, knee, spine, tibia, etc.). Various drill bits, screws, pins, reamers, and anchors exit out of the nose cone 32. Although the nose cone 32 is shown to have a conical shape, other shapes (e.g., cylindrical) are also possible. The nose cone 32 is sometimes referred to as a cannula.

The rail 34 is positioned on the support portion 17 in the housing 12. The rail 34 is a continuous bar that acts as a guide for the carriage 36. The carriage 36 is configured to glide along the rail 34. The rail 34 includes a rail platform 37 that is engaged with the rail 34 and configured to slide along the rail 34 in the forward direction 52 and the backward direction 54 without decoupling from the rail 34.

The carriage 36 is coupled to the rail 34, more specifically the rail platform 37. The carriage 36 is secured to the rail platform 37 and is able to slide along the rail 34 without decoupling from the rail 34. The carriage 36 provides a support for the chuck 46 and the drill motor 48. The carriage 36 includes a threaded portion 56 and a mounting portion 57. The threaded portion 56 mates with the lead screw 38. More specifically, the threaded portion 56 includes internal threads that are mated with external threads of the lead screw 38. The mounting portion 57 provides a support for the chuck 46 and the drill motor 48.

The lead screw 38 is coupled to the carriage 36, the bearing structure 40, and the shaft coupling 42. The lead screw 38 extends through the threaded portion 56, and extends between the bearing structure 40 and the shaft coupling 42. The lead screw 38 is a threaded rod having external threads that are mated with the threaded portion 56. The lead screw 38 is configured to rotate around an axis 58 (referring to FIG. 3). As the external threads are mated with the internal threads of the threaded portion 56, the rotation of the lead screw 38 by the linear motor 44 cause the carriage 36 to move along the axis 58.

The bearing structure 40 is coupled between a sidewall of the housing 12 and a first end of the lead screw 38. The bearing structure 40 couples the leading screw 38 to the housing 12 while also allowing the lead screw 38 to freely rotate around the axis 58. In one embodiment, the bearing structure 40 is a ball bearing.

The shaft coupling 42 is coupled between a second end, opposite to the first end, of the lead screw 38 and the linear motor 44. The shaft coupling 42 is a mechanical coupling that connects the linear motor 44 to the lead screw 38 and transmits power from the linear motor 44 to the lead screw 38. Stated differently, the shaft coupling 42 transfers the rotation motion generated by the linear motor 44 directly to the lead screw 38. As a result, the linear motor 44 is able to rotate the lead screw 38 around the axis 58. The shaft coupling 42, for example, minimizes misalignment and vibration between the linear motor 44 and the lead screw 38 and ensures proper rotation of the lead screw 38.

The linear motor 44 is coupled to the lead screw 38 by the shaft coupling 42. The linear motor 44 rotates the shaft coupling 42, which in turn rotates the lead screw 38 around the axis 58. The linear motor 44 may be any type of motor that provides a rotating motion for the lead screw 38.

The linear motor 44 includes a motor encoder that detects a determined rotation or step amount (e.g., 5, 10, 15, …, or 360 degrees) of the linear motor 44 around the axis 58. The motor encoder translates each determined rotation amount of the linear motor 44 into an electrical signal or pulse, and transmits the electrical signal to the linear controller 26 and/or the application controller 30 for further processing. As will be discussed in further detail below, a total step count of the determined rotation amounts is used to determine a depth measurement of a current hole being drilled by the device 10.

The chuck 46 is positioned on the carriage 36, more specifically on the mounting portion 57. The chuck 46 is a bit mount configured to hold or clamp a tool, such as a drill bit, a screw, a pin, a reamer, and an anchor, to be used in a drill and implantation process. The chuck 46 rotates around an axis 62 (referring to FIG. 3). The axis 62 is parallel to the axis 58.

The drill motor 48 is positioned on the carriage 36, more specifically on the mounting portion 57, along with the chuck 46. The drill motor 48 extends through a through hole of the mounting portion 57. The drill motor 48 rotates the chuck 46 around the axis 62. As the drill motor 48 rotates the chuck 46, the tool loaded in the chuck 46 rotates around the axis 62. For example, as the drill motor 48 rotates the chuck 46, the drill bit 11 rotates around the axis 62 to drill. The drill motor 48 may be any type of motor that provides a rotating motion for the chuck 46. The drill motor 48 is able to operate concurrently with the linear motor 44.

Although the drill motor 48 and the linear motor 44 are shown as separate motors, the functions of the drill motor 48 and the linear motor 44 may also be combined and performed by a single motor using, for example, switching couplings and/or gear shifters.

In one embodiment, the linear motor 44 and the drill motor 48 include torque restrictors configured to prevent overtightening an implant and to prevent excessive torque on and damage to the linear motor 44 and the drill motor 48.

As best shown in FIG. 5, the linear motor 44 is coupled to the lead screw 38 by the shaft coupling 42, and the bearing structure 40 and the shaft coupling 42 are coupled to the threaded portion 56 of the carriage 36 by the lead screw 38. The lead screw 38 in turn is coupled to the rail 34 by the carriage 36. Further, the chuck 46 and the drill motor 48 are coupled to the mounting portion 57 of the carriage 36.

With this configuration, when the linear motor 44 rotates the lead screw 38, the carriage 36 moves along the axis 58. As a result, the chuck 46, the drill motor 48, and the tool loaded in the chuck 46 are able to move in the forward direction 52 and the backward direction 54 while remaining components of the device 10 (e.g., the housing 12, the handle 14, the battery 15, the upper trigger 16, the lower trigger 18, the user interface 20, the controller cover 22, the linear controller 26, the drill controller 28, the application controller 30, the nose cone 32, the rail 34, the lead screw 38, the bearing structure 40, the shaft coupling 42, and the linear motor 44) remain stationary with respect to the handle 14.

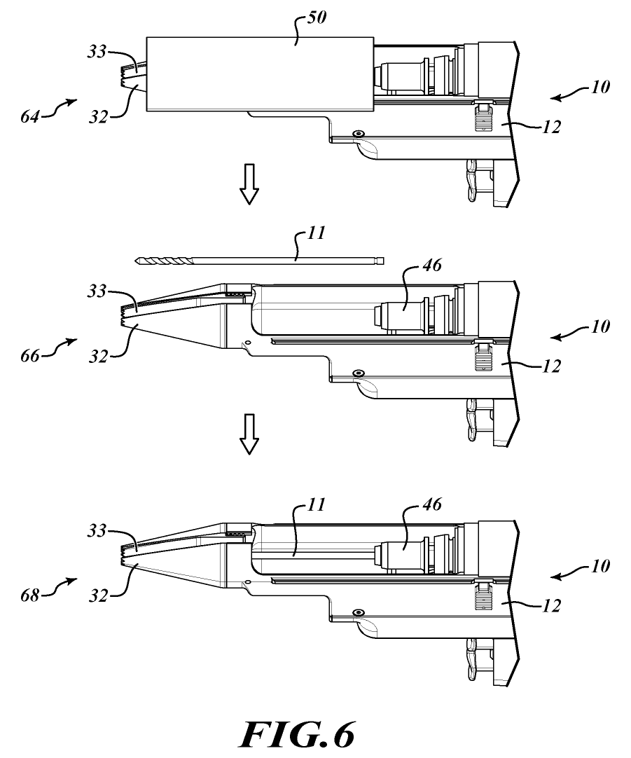

FIG. 6 shows a loading of, for example, the drill bit 11 into the device 10 according to an embodiment disclosed herein. Although the drill bit 11 is shown as being loaded into the device 10, other types of tools, such as screws, pins, reamers, and anchors, are similarly loaded into the device 10.

In a first step 64, the door 50 is removed from the housing 12. As a result, the chuck 46 and the guide portion 33 of the nose cone 32 are exposed. The door 50 may also be configured to open (e.g., flip up) instead of being removed.

In a second step 66, the drill bit 11 is positioned in the housing 12 by inserting the drill bit 11 in the guide portion 33 of the nose cone 32.

In a third step 68, the drill bit 11 is loaded into the chuck 46. The drill bit 11 is aligned with the chuck 46 and extends from the chuck 46 and into the guide portion 33 of the nose cone 32. Once the drill bit 11 is secured by the chuck 46, the door 50 is closed and a drill and implantation process may then be performed.

The device 10 is configured to operate in a plurality of different operation modes: a manual mode, a first cortex drill mode, a second cortex find mode, a second cortex drill mode, a fixed depth mode, and a lag screw mode. The device 10 drills into a target bone to different depths depending on the operation mode. The user selects one of the operation modes using the user interface 20. The operation modes are executed by the application controller 30, which in turn controls the various components of the device 10.

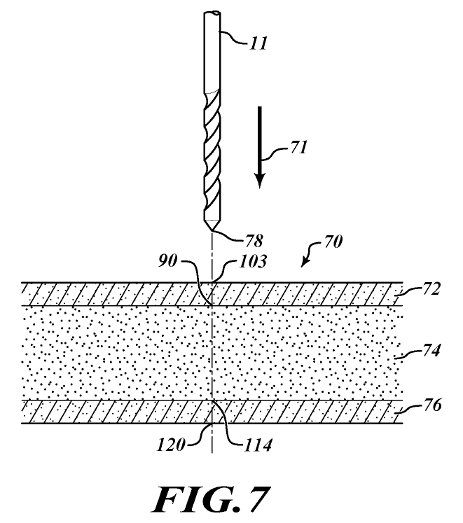

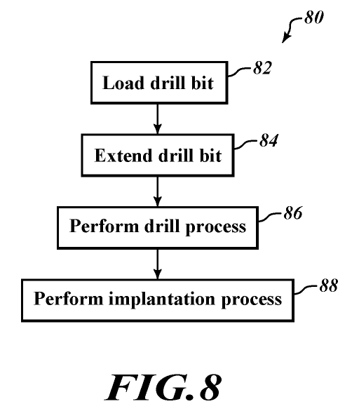

FIG. 7 shows a target bone 70 according to an embodiment disclosed herein. The bone 70 includes a near cortex 72, an intramedullary cavity 74, and a far cortex 76. The drill bit 11 has a tip 78 and is used to drill into the bone 70 in a drill direction 71. FIG. 7 will be referenced as an example during the discussion of FIGS. 8-13 below.

FIG. 8 is a block diagram of a method 80 of operating a manual mode of the device 10 according to an embodiment disclosed herein. In the manual mode, the device 10 extends the drill bit 11 out of the nose cone 32, and the user manually moves the device 10 to drill into the target bone 70 to a determined depth. Stated differently, the drill bit 11 is not automatically moved in the forward direction 52 or the backward direction 54 by the linear motor 44.

In block 82, the drill bit 11 is loaded into the device 10. The drill bit 11 is loaded into the device 10 as discussed with respect to FIG. 6. The user confirms that the drill bit 11 is loaded using the user interface 20.

In block 84, the drill bit 11 is moved to extend out of the nose cone 32. More specifically, the application controller 30 instructs the linear controller 26 to control the linear motor 44 to rotate the lead screw 38 (through the shaft coupling 42) such that the carriage 36 moves in the forward direction 52. The drill bit 11, the chuck 46, and the drill motor 48 move along with the carriage 36. The carriage is moved until the drill bit 11 extends out of the nose cone 32.

In block 86, the device 10 is used by the user manually to perform a drill process. The user manually moves the device 10 to drill into the target bone 70 to a determined depth. The drill bit 11 is not automatically moved in the forward direction 52 or the backward direction 54 by the linear motor 44.

In the manual mode, the upper trigger 16 operates the drill motor 48 to rotate the chuck 46 and the drill bit 11 in a clockwise direction, and the lower trigger 18 operates the drill motor 48 to rotate the chuck 46 and the drill bit 11 in counter-clockwise direction (or vice versa). The speed of the drill motor 48 is controlled by the amount of pressure on the upper trigger 16 and the lower trigger 18.

In block 88, the device 10 is used to perform an implantation process. In the implantation process, the drill bit 11 is unloaded from the chuck 46. The drill bit 11 is removed by removing the door 50 from the housing 12, and removing the drill bit 11 from the chuck 46. An implant driver with an implant, such as a screw, screw holder, a pin, or a pin holder, is then loaded into the device 10 as discussed with respect to FIG. 6. The user confirms that the implant is loaded using the user interface 20. The device 10 is then used by the user to manually insert the implant into the target bone 70 by, for example, depressing one of the upper trigger 16 or the lower trigger 18. The user manually moves the device 10 to insert the implant into the target bone 70 to a determined depth. The implant is not automatically moved in the forward direction 52 or the backward direction 54 by the linear motor 44.

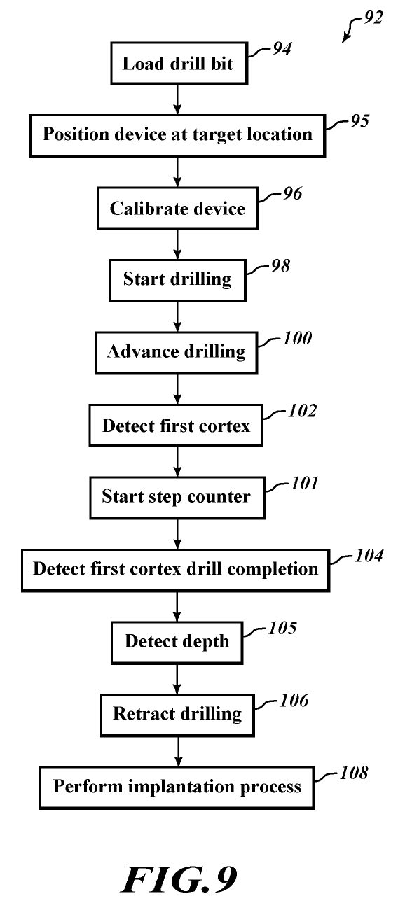

FIG. 9 is a block diagram of a method 92 of operating a first cortex drill mode of the device 10 according to an embodiment disclosed herein. In the first cortex drill mode, the device 10 automatically drills through the first, near cortex 72 and stops once the intramedullary cavity 74 is reached. More specifically, the drill process is stopped when the tip 78 of the drill bit 11 at or passed position 90 (referring to FIG. 7).

In block 94, the drill bit 11 is loaded into the device 10. The drill bit 11 is loaded into the device 10 as discussed with respect to FIG. 6. The user confirms that the drill bit 11 is loaded using the user interface 20.

In block 95, the device 10 is positioned at a target location, such as the bone 70, by the user. The device 10 is positioned such that the nose cone 32 is in physical contact with the bone 70 and the opening of the nose cone 32 directly overlies the bone 70. Once the device 10 is put into position in block 95, the device 10 remains stationary with respect to the handle 14 for the remainder of the method 92. Further, the user does not apply physical force to push the device 10 to drill or insert an implant into the bone. As a result, fatigue of the user is minimized.

In block 96, the device 10 is calibrated for subsequent torque measurements. During calibration, the drill bit 11 is moved into a calibration position in which the tip 78 of the drill bit 11 is in the nose cone 32 but does not extend out of the opening of the nose cone 32. The tip 78 of the drill bit 11 is spaced from the opening of the nose cone 32 by a determined distance (e.g., 5-10 millimeters). As a result, the drill bit 11 is not physically contacting the bone 70.

In one embodiment, the user manually moves the tip 78 into the calibration position using the user interface 20, the upper trigger 16, and/or the lower trigger 18. For example, the user pulls both the upper trigger 16 and the lower trigger 18 concurrently to initiate the calibration.

In one embodiment, the application controller 30 instructs the linear controller 26 to control the linear motor 44 to rotate the lead screw 38 (through the shaft coupling 42) such that the carriage 36, along with the drill bit 11, the chuck 46, and the drill motor 48, moves in the forward direction 52. The carriage is moved until the drill bit 11 is in the calibration position. In this embodiment, the user enters parameters of the drill bit 11 (e.g., type, length, pitch, etc.) so that the application controller 30 is able to determine when the drill bit 11 is in the calibration position based on the parameters.

Once the drill bit 11 is in the calibration position, the application controller 30 instructs the drill motor 48 to rotate the chuck 46 and the drill bit 11, and measures the torque of the linear motor 44, the drill motor 48, or both the linear motor 44 and the drill motor 48. As there is no contact between the drill bit 11 and the bone 70, the measured torque provides a baseline or reference torque value when there is no physical load on the device 10. The application controller 30 measures torque using, for example, a torque sensor or a combination of force and distance sensors, or estimates torque based on, for example, electrical parameters (e.g., electrical current or power draw) of the linear motor 44 and/or the drill motor 48.

The application controller 30 continuously measures the torque of the linear motor 44, the drill motor 48, or both the linear motor 44 and the drill motor 48 throughout blocks 98, 100, 102, and 104.

Although block 96 is shown subsequent to block 95 in FIG. 9, block 96 may also be performed prior to or concurrent with block 95. For example, the device 10 is calibrated in block 96 prior to the device 10 being positioned at the target location in block 95.

In block 98, drilling by the device 10 is started. The application controller 30 instructs the drill controller 28 to start the drill motor 48, which in turn rotates the chuck 46 and the drill bit 11 in a first direction (e.g., clockwise). In one embodiment, the drill motor 48 is ramped up to its maximum RPM.

In block 100, the drilling by the device 10 is advanced concurrently with the drilling started in block 98. The application controller 30 instructs the linear controller 26 to rotate the linear motor 44 in a first direction (e.g., clockwise), which in turn rotates the lead screw 38 (through the shaft coupling 42). As a result, the carriage 36, along with the drill bit 11, the chuck 46, and the drill motor 48, moves in the forward direction 52. In one embodiment, the carriage 36 is advanced at a constant rate. In one embodiment, the carriage 36 is advanced at a variable rate dependent on the power consumption of the device 10. For example, the carriage 36 is advanced at a slower rate when power consumption of, for example, the linear motor 44 and/or the drill motor 48 is greater than a determined threshold.

In block 102, the application controller 30 detects the first cortex 72. More specifically, the application controller 30 detects that the tip 78 of the drill bit 11 contacts the first cortex 72 at position 103 (referring to FIG. 7) based on the measured torque of the linear motor 44, the drill motor 48, or both the linear motor 44 and the drill motor 48.

In one embodiment, the application controller 30 detects the drill bit 11 has contacted the first cortex 72 in response to determining the measured torque of the linear motor 44, the drill motor 48, or both the linear motor 44 and the drill motor 48 has increased from their respective baseline torque value in block 96 by a determined amount.

In one embodiment, the application controller 30 detects the drill bit 11 has contacted the first cortex 72 in response to determining the measured torque of the linear motor 44, the drill motor 48, or both the linear motor 44 and the drill motor 48 has increased from their respective baseline torque value in block 96 by a determined amount and a determined rate of increase.

The application controller 30 saves and sets the increased torque value (i.e., the current torque value) as a first cortex baseline torque value. In addition, the detection of the first cortex 72 is displayed on the user interface 20. The drilling by the device 10 is then continued to be linearly advanced concurrently with the drilling as discussed with respect to block 100.

In block 101, a step counter is started. As discussed above, the linear motor 44 includes a motor encoder that detects a determined rotation or step amount (e.g., 5, 10, 15, …, or 360 degrees) of the linear motor 44. The motor encoder translates each determined rotation amount of the linear motor 44 into an electrical signal or pulse, and transmits the electrical signal to the linear controller 26 and/or the application controller 30. In block 101, the step counter, which is initialized to zero, is started in response to detecting the first cortex 72 in block 102. The step counter is incremented each time the electrical signal is received from the motor encoder. Stated differently, after the first cortex 72 is detected, the step counter is incremented each time the motor encoder detects the determined rotation amount. As such, the step counter represents a total count of the determined rotation amount detected since the first cortex 72 was detected. In one embodiment, the application controller 30 starts and keeps track of the step counter. In one embodiment, the linear controller 26 starts and keeps track of the step counter, and reports the step counter to the application controller 30 for further processing. The incrementing and tracking of the step counter are continued once the step counter is started. The drilling by the device 10 is also continued to be linearly advanced concurrently with the drilling as discussed with respect to block 100.

In block 104, the application controller 30 detects that the drilling of the first cortex 72 has completed and the tip 78 of the drill bit 11 has reached or passed position 90 at the intramedullary cavity 74 based on the measured torque of the linear motor 44, the drill motor 48, or both the linear motor 44 and the drill motor 48.

In one embodiment, the application controller 30 detects the drill bit 11 has reached the intramedullary cavity 74 in response to determining the measured torque of the linear motor 44, the drill motor 48, or both the linear motor 44 and the drill motor 48 has decreased from their respective first cortex baseline torque value in block 102 by a determined amount.

In one embodiment, the application controller 30 detects the drill bit 11 has reached the intramedullary cavity 74 in response to determining the measured torque of the linear motor 44, the drill motor 48, or both the linear motor 44 and the drill motor 48 has decreased from their respective first cortex baseline torque value in block 102 by a determined amount and at a determined rate of decrease.

The completion of the drilling of the first cortex 72 and the detection of the intramedullary cavity 74 are displayed on the user interface 20.

In block 105, the application controller 30 determines a current depth of the hole currently being drilled. The current depth is the distance the tip 78 of the drill bit 11 has traveled into the bone 70 in the drill direction 71, starting from the position 103 (where the tip 78 of the drill bit 11 first contacts the first cortex 72).

As block 105 is performed in response to detection that the drilling of the first cortex 72 has completed in block 104, the current depth indicates the depth of the hole drilled from position 103 to position 90.

The application controller 30 determines the current depth based on parameters of the lead screw 38 and the current value of the step counter in block 101. The parameters of the lead screw 38 includes various parameters of the lead screw 38, such as type of material, type of lead screw, a length of the lead screw, a pitch of the lead screw, and a lead of the lead screw. The pitch of the lead screw 38 indicates a distance between adjacent threads of the lead screw 38. The lead of the lead screw 38 indicates a linear distance along the axis 58 the lead screw 38 travels per determined rotation (e.g., 360 degrees) of the lead screw 38. The parameters of the lead screw 38 are entered into the device 10 through the user interface 20, and may be entered at any time prior to block 105.

In one embodiment, the current depth is determined based on the pitch of the lead screw 38 and the current value of the step counter. The pitch of the lead screw 38 indicates a distance between adjacent threads of the lead screw 38. For example, the application controller 30 determines the pitch of the lead screw 38 corresponds to a determined depth distance per determined rotation amount, and determines the current depth based on the product of the current value of the step counter and the determined depth distance per determined rotation amount.

Similarly, in one embodiment, the current depth is determined based on the lead of the lead screw 38 and the current value of the step counter. The lead of the lead screw 38 indicates a linear distance along the axis 58 the lead screw 38 travels in a determined rotation (e.g., 360 degrees) of the lead screw 38. For example, the application controller 30 determines the lead of the lead screw 38 corresponds to a determined depth distance per determined rotation amount, and determines the current depth based on the product of the current value of the step counter and the determined depth distance per determined rotation amount.

It is noted that the determined current depth of the drill in block 105 is specific to the lead screw 38 because depth calculations are based on the parameters of the lead screw 38. If, for example, a new lead screw 38 is used, new parameters will need to be entered for an accurate calculation in block 105.

In block 106, the drilling of the device 10 is retracted. The application controller 30 instructs the drill controller 28 to stop and then reverse the direction of the drill motor 48, which in turn rotates the chuck 46 and the drill bit 11 in a second direction (e.g., counter-clockwise). Concurrently, the application controller 30 instructs the linear controller 26 to stop and reverse rotation of the linear motor 44, which in turn rotates the lead screw 38 (through the shaft coupling 42) in a second direction (e.g., counter-clockwise). As a result, the carriage 36, along with the drill bit 11, the chuck 46, and the drill motor 48, moves in the backward direction 54. In one embodiment, the carriage 36 is retracted at a constant rate. In one embodiment, the carriage 36 is retracted at a variable rate dependent on the power consumption of the linear motor 44. The drilling of the device 10 is retracted to a determined retracted position until the drill bit 11 is returned back into the nose cone 32, and, thus, no longer contacting the bone 70 (e.g., back into the calibration position).

In block 108, the device 10 is used to perform an implantation process. In the implantation process, the drill bit 11 is unloaded from the chuck 46. The drill bit 11 is removed by removing the door 50 from the housing 12, and removing the drill bit 11 from the chuck 46. An implant driver with an implant, such as a screw, screw holder, a pin, or a pin holder, is then loaded into the device 10 as discussed with respect to FIG. 6. The user confirms that the implant is loaded using the user interface 20. The device 10 is then used by the user to automatically insert the implant into the bone 70.

The implant is selected based on the depth determined in block 105. For example, an implant with a length corresponding to or equal to the depth determined in block 105 is selected. In one embodiment, the application controller 30 determines the implant based on the depth determined in block 105, and displays the determined implant on the user interface 20.

In one embodiment, the determined retracted position is a position in which a tip of the implant is located at position 103.

To insert the implant into the bone 70, the application controller 30 instructs the drill controller 28 to start the drill motor 48, which in turn rotates the chuck 46 and the implant in a first direction (e.g., clockwise). While the implant is rotating in the first direction, the application controller 30 instructs the linear controller 26 to rotate the linear motor 44 in a first direction (e.g., clockwise), which in turn rotates the lead screw 38 (through the shaft coupling 42). As a result, the carriage 36, along with the implant, the chuck 46, and the drill motor 48, moves in the forward direction 52.

The rotating and the advancement of the implant is continued until the implant is fully inserted into the bone 70. In one embodiment, the application controller 30 detects the implant is fully inserted in response to determining the implant has been inserted to the depth determined in block 105.

The application controller 30 determines the depth of the implant with the same techniques as discussed with respect to block 105. Namely, the application controller 30 determines the current depth of the implant based on parameters of the lead screw 38 and the current value of the step counter. In this case, however, the step counter is initialized to zero once the implant is loaded into the device 10 at the determined retracted position, and the step counter is started in response to the application controller 30 instructing the drill motor 48 and the linear controller 26 to start to insert the implant into the bone 70. The current depth of the implant is then determined based on the pitch or the lead of the lead screw 38 and the current value of the step counter, as discussed above. The application controller 30 determines the implant has been inserted when the current depth corresponds to or is equal to the depth determined in block 105.

The insertion of the implant is displayed on the user interface 20. Once the implant is fully inserted into the bone 70, the implant driver is disengaged from the implant by stopping the drill motor 48 so that the implant driver is able to retract without rotating the implant out. The device 10 is then retracted again as discussed with respect to block 106.

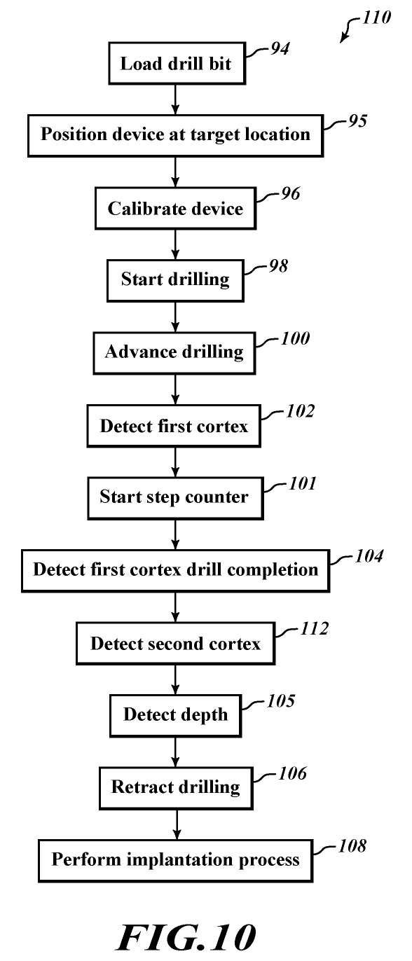

FIG. 10 is a block diagram of a method 110 of operating a second cortex find mode of the device 10 according to an embodiment disclosed herein. In the second cortex find mode, the device 10 automatically drills through the first, near cortex 72, through the intramedullary cavity 74, and stops once the second, far cortex 76 is reached. More specifically, the drill process is stopped when the tip 78 of the drill bit 11 is at position 114 (referring to FIG. 7).

In the method 110, blocks 94, 95, 96, 98, 100, 102, 101, and 104 are performed as discussed above with respect to FIG. 9. In block 94, the drill bit 11 is loaded into the device 10. In block 95, the device 10 is positioned at a target location, such as the bone 70, by the user. In block 96, the device 10 is calibrated. In block 98, drilling by the device 10 is started. In block 100, the drilling by the device 10 is advanced concurrently with the drilling started in block 98. In block 102, the application controller 30 detects that the tip 78 of the drill bit 11 contacts the first cortex 72 at position 103. In block 101, a step counter is started. In block 104, the application controller 30 detects that the drilling of the first cortex 72 has completed and the tip 78 of the drill bit 11 has reached position 90 at the intramedullary cavity 74.

As discussed above, in block 104, the application controller 30 detects that the drilling of the first cortex 72 has completed and the tip 78 of the drill bit 11 has reached position 90 at the intramedullary cavity 74 based on the measured torque of the linear motor 44, the drill motor 48, or both the linear motor 44 and the drill motor 48. In one embodiment, the application controller 30 detects the drill bit 11 has reached the intramedullary cavity 74 in response to determining the measured torque of the linear motor 44, the drill motor 48, or both the linear motor 44 and the drill motor 48 has decreased from their respective first cortex baseline torque value in block 102 by a determined amount. In one embodiment, the application controller 30 detects the drill bit 11 has reached the intramedullary cavity 74 in response to determining the measured torque of the linear motor 44, the drill motor 48, or both the linear motor 44 and the drill motor 48 has decreased from their respective first cortex baseline torque value in block 102 by a determined amount and at a determined rate of decrease. The completion of the drilling of the first cortex 72 and the detection of the intramedullary cavity 74 are displayed on the user interface 20.

In addition, in block 104 of the method 110, the application controller 30 saves and sets the decreased torque value (i.e., the current torque value) as an intramedullary cavity baseline torque value. The drilling by the device 10 is then continued to be linearly advanced concurrently with the drilling as discussed with respect to block 100.

In block 112, the application controller 30 detects the second cortex 76. More specifically, the application controller 30 detects that the tip 78 of the drill bit 11 contacts the second cortex 76 at position 114 based on the measured torque of the linear motor 44, the drill motor 48, or both the linear motor 44 and the drill motor 48. It is noted that the drill bit 11 scores the second cortex 76 at position 114 but does not penetrate the second cortex 76.

In one embodiment, the application controller 30 detects the drill bit 11 has contacted the second cortex 76 in response to determining the measured torque of the linear motor 44, the drill motor 48, or both the linear motor 44 and the drill motor 48 has increased from their respective intramedullary cavity baseline torque value in block 104 by a determined amount.

In one embodiment, the application controller 30 detects the drill bit 11 has contacted the second cortex 76 in response to determining the measured torque of the linear motor 44, the drill motor 48, or both the linear motor 44 and the drill motor 48 has increased from their respective intramedullary cavity baseline torque value in block 104 by a determined amount and at a determined rate of decrease.

The detection of the second cortex 76 is displayed on the user interface 20.

As discussed above, in block 105, the application controller 30 determines a current depth of the hole currently being drilled. The current depth is the distance the tip 78 of the drill bit 11 has traveled into the bone 70 in the drill direction 71, starting from the position 103 (where the tip 78 of the drill bit 11 first contacts the first cortex 72). As block 105 of the method 110 is performed in response to detection of the second cortex 76 in block 112, the current depth indicates the depth of the hole drilled from position 103 to position 114. The current depth of the hole currently being drilled is determined based on the pitch or the lead of the lead screw 38 and the current value of the step counter, as discussed above.

Blocks 106 and 108 are then performed as discussed above. In block 106, the drilling of the device 10 is retracted. In block 108, the device 10 is used to perform an implantation process.

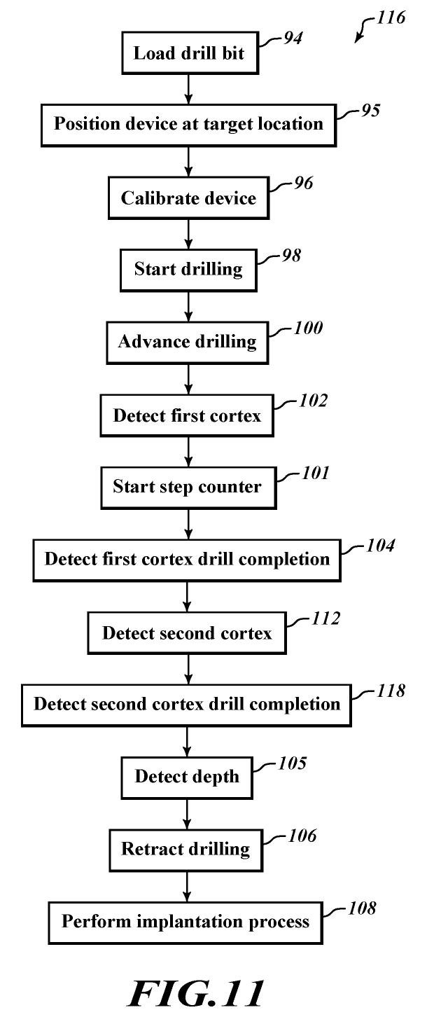

FIG. 11 is a block diagram of a method 116 of operating a second cortex drill mode of the device 10 according to an embodiment disclosed herein. In the second cortex drill mode, the device 10 automatically drills through the first, near cortex 72, through the intramedullary cavity 74, through the second cortex 76, and stops after the second cortex 76 has been drilled through. More specifically, the drill process is stopped when the tip 78 of the drill bit 11 is at or passed position 120 (referring to FIG. 7).

In the method 116, blocks 94, 95, 96, 98, 100, 102, and 101, are performed as discussed above with respect to FIG. 9 and blocks 104 and 112 are performed as discussed above with respect to FIG. 10. In block 94, the drill bit 11 is loaded into the device 10. In block 95, the device 10 is positioned at a target location, such as the bone 70, by the user. In block 96, the device 10 is calibrated. In block 98, drilling by the device 10 is started. In block 100, the drilling by the device 10 is advanced concurrently with the drilling started in block 98. In block 102, the application controller 30 detects that the tip 78 of the drill bit 11 contacts the first cortex 72 at position 103. In block 101, a step counter is started. In block 104, the application controller 30 detects that the drilling of the first cortex 72 has completed and the tip 78 of the drill bit 11 has reached position 90 at the intramedullary cavity 74. In block 112, the application controller 30 detects the second cortex 76.

As discussed above, in block 112, the application controller 30 detects the second cortex 76. More specifically, the application controller 30 detects that the tip 78 of the drill bit 11 contacts the second cortex 76 at position 114 based on the measured torque of the linear motor 44, the drill motor 48, or both the linear motor 44 and the drill motor 48. In one embodiment, the application controller 30 detects the drill bit 11 has contacted the second cortex 76 in response to determining the measured torque of the linear motor 44, the drill motor 48, or both the linear motor 44 and the drill motor 48 has increased from their respective intramedullary cavity baseline torque value in block 104 by a determined amount. In one embodiment, the application controller 30 detects the drill bit 11 has contacted the second cortex 76 in response to determining the measured torque of the linear motor 44, the drill motor 48, or both the linear motor 44 and the drill motor 48 has increased from their respective intramedullary cavity baseline torque value in block 104 by a determined amount and at a determined rate of decrease. The detection of the second cortex 76 is displayed on the user interface 20.

In addition, in block 112 of the method 116, the application controller 30 saves and sets the increased torque value (i.e., the current torque value) as a second cortex baseline torque value. The drilling by the device 10 is then continued to be linearly advanced concurrently with the drilling as discussed with respect to block 100.

In block 118, the application controller 30 detects that the drilling of the second cortex 76 has completed and the tip 78 of the drill bit 11 has reached or passed position 120 based on the measured torque of the linear motor 44, the drill motor 48, or both the linear motor 44 and the drill motor 48.

In one embodiment, the application controller 30 detects the drill bit 11 has finished drilling the second cortex 76 in response to determining the measured torque of the linear motor 44, the drill motor 48, or both the linear motor 44 and the drill motor 48 has decreased from their respective second cortex baseline torque value in block 112 by a determined amount.

In one embodiment, the application controller 30 detects the drill bit 11 has finished drilling the second cortex 76 in response to determining the measured torque of the linear motor 44, the drill motor 48, or both the linear motor 44 and the drill motor 48 has decreased from their respective second cortex baseline torque value in block 112 by a determined amount and at a determined rate of decrease.

The completion of the drilling of the second cortex 76 is displayed on the user interface 20.

As discussed above, in block 105, the application controller 30 determines a current depth of the hole currently being drilled. The current depth is the distance the tip 78 of the drill bit 11 has traveled into the bone 70 in the drill direction 71, starting from the position 103 (where the tip 78 of the drill bit 11 first contacts the first cortex 72). As block 105 of the method 116 is performed in response to detection that the drilling of the second cortex 76 has completed in block 118, the current depth indicates the depth of the hole drilled from position 103 to position 120. The current depth of the hole currently being drilled is determined based on the pitch or the lead of the lead screw 38 and the current value of the step counter, as discussed above.

Blocks 106 and 108 are then performed as discussed above. In block 106, the drilling of the device 10 is retracted. In block 108, the device 10 is used to perform an implantation process.

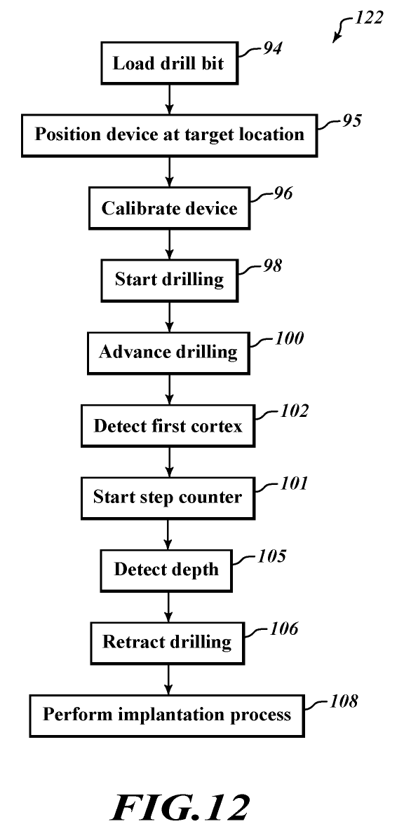

FIG. 12 is a block diagram of a method 122 of operating a fixed depth mode of the device 10 according to an embodiment disclosed herein. In the fixed depth mode, the device 10 automatically drills to a fixed, target depth into the bone 70 and stops once the fixed depth is reached. The fixed depth may be any depth past the position 103.

In the method 122, blocks 94, 95, 96, 98, 100, 102, 101, and 105 are performed as discussed above with respect to FIG. 9. In block 94, the drill bit 11 is loaded into the device 10. In block 95, the device 10 is positioned at a target location, such as the bone 70, by the user. In block 96, the device 10 is calibrated. In block 98, drilling by the device 10 is started. In block 100, the drilling by the device 10 is advanced concurrently with the drilling started in block 98. In block 102, the application controller 30 detects that the tip 78 of the drill bit 11 contacts the first cortex 72 at position 103. In block 101, a step counter is started. In block 105, the application controller 30 determines a current depth of the hole currently being drilled.

As block 105 of the method 122 is performed in response to detection of the first cortex in block 102, the current depth indicates the depth of the hole drilled starting from position 103. The current depth of the hole currently being drilled is determined based on the pitch or the lead of the lead screw 38 and the current value of the step counter, as discussed above.

The drilling by the device 10 is continued to be linearly advanced concurrently with the drilling, while the application controller 30 continuously determines the current depth of the drill. The current depth is displayed on the user interface 20. The method 122 moves to block 106 in response to the current depth being equal to the fixed, target depth.

Blocks 106 and 108 are then performed as discussed above. In block 106, the drilling of the device 10 is retracted. In block 108, the device 10 is used to perform an implantation process.

As the device 10 is able to obtain an accurate depth measurement in block 105, the user is able to select an implant in block 108 with the correct length to subsequently insert into the patient, and, thus, avoiding the use of implants with the incorrect length and reducing hardware waste, costs, and time.

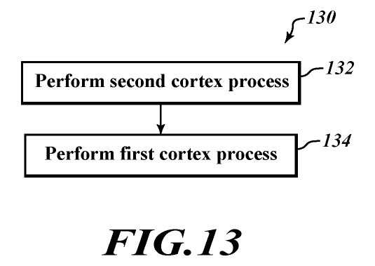

FIG. 13 is a block diagram of a method 130 of operating a lag screw mode of the device 10 according to an embodiment disclosed herein. In the lag screw mode, the device 10 performs the second cortex drill mode with a first drill bit, and subsequently performs the first cortex drill mode with a second drill bit having a greater thickness (e.g., diameter) than the first drill bit.

In block 132, the second cortex drill mode is performed as discussed with respect to FIG. 11. However, referring to FIG. 11, a first drill bit having a first thickness (e.g., dimension of the drill bit 11 transverse to the drill direction 71) is loaded in block 94. In addition, block 108, in which the device 10 is used to perform an implantation process, is not performed. The method 130 moves to block 134 after block 106 is completed.

In block 134, the first cortex drill mode is performed as discussed with respect to FIG. 9. However, referring to FIG. 9, a second drill bit having a second thickness greater than the first thickness is loaded in block 94.

With respect to the methods 92, 110, 116, 122, and 130 discussed above, the device 10 may remain stationary once the device 10 is positioned at the target location. Because of this, the device 10 has the capability of attaching radiolucent components to the device 10 so that the device 10 may drill, measure, and insert implants either remotely or manually outside a direct field of an x-ray or fluoroscopy beam.

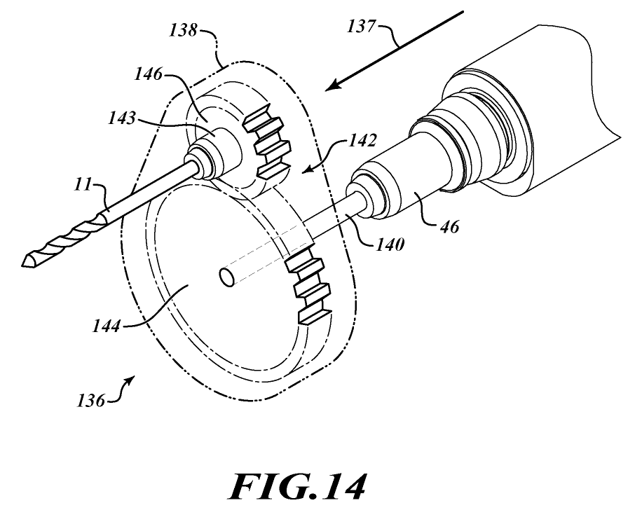

FIG. 14 is a radiolucent attachment 136 according to an embodiment disclosed herein. The radiolucent attachment 136 attaches to the device 10 and allows the device 10 to be positioned outside a direct field of an x-ray or fluoroscopy beam 137. The radiolucent attachment 136 is mounted with the drill bit 11 in FIG. 14. However, the device 10 may be mounted with various tools, such as drill bits, screws, pins, reamers, and anchors. The nose cone 32 is not shown in FIG. 14 for simplicity.

The radiolucent attachment 136 includes a housing 138, a connector 140, gears 142, and a secondary chuck 143.

The housing 138 is a protective body that contains internal components of the radiolucent attachment 136.

The connector 140 extends in and out of the housing 138. The connector 140 is a shaft that is used to physically attach the radiolucent attachment 136 to the device 10. The connector 140 is configured to be inserted into the chuck 46 like other bits. Once inserted, the chuck 46 holds or clamps the connector 140. As a result, the connector 140 rotates as the chuck 46 rotates.

The gears 142 are in the housing 138. The gears 142 are coupled together and transmit the rotational movement from the chuck 46 and the connector 140 to the drill bit 11. The gears 142 include a first gear 144 and a second gear 146. Although two gears are shown in FIG. 14, the radiolucent attachment 136 may include any number of gears.

The connector 140 is physically attached to or inserted into a central portion of the first gear 144. As a result, the first gear 144 rotates as the connector 140 rotates. In one embodiment, the connector 40 and the first gear 144 are one contiguous component.

The second gear 146 is coupled to the first gear 144. The teeth of the second gear 146 are meshed or engaged with the teeth of the first gear 144. As a result, the second gear 146 rotates as the first gear 144 rotates.

The secondary chuck 143 is accessible outside of the housing 138. The secondary chuck 143 is physically attached to or inserted into a central portion of the second gear 146. Similar to the chuck 46, the secondary chuck 143 is a bit mount configured to hold or clamp a tool, such as a drill bit, a screw, and a pin, to be used in a drill and implantation process. The secondary chuck 143 rotates as the second gear 146 rotates. As shown in FIG. 14, the secondary chuck 143, through the use of the gears 142, is positioned vertically higher than the chuck 46.

In one embodiment, the radiolucent attachment 136 does not include the secondary chuck 143, and the drill bit 11 is directly mounted on the central portion of the second gear 146 such that the drill bit 11 and the second gear 146 are one contiguous component.

The radiolucent attachment 136 is made of radiolucent materials to allow x-rays, such as the x-ray beam 137, to pass through. More specifically, the housing 138, the connector 140, the gears 142, and the secondary chuck 143 are made of radiolucent materials. Radiolucent materials may include plastic or carbon fiber. In one embodiment, components of the radiolucent attachment 136, which will not be aligned with x-rays, such as the x-ray beam 137, are not made of radiolucent materials. For example, the connector 140 and the first gear 144 may not be made of radiolucent materials.

With the secondary chuck 143 positioned vertically higher than the chuck 46 and the use of radiolucent materials, the drill bit 11 may be positioned at the target position where the x-ray beam 137 is transmitted without the device 10, which may include non-radiolucent materials, interfering with the x-ray beam 137. As such, x-ray images of, for example, the drill bit 11 and the target bone may be easily obtained during performance of the methods 92, 110, 116, 122, and 130, without the device 10 blocking the views.

In one embodiment, the drill bit 11 is radiolucent with a radioopaque component (e.g., a bead of metal) at the tip of the drill bit 11. As a result, radiographic visualization of the position of the tip of the drill bit 11 is easily obtained during certain surgical procedures, such as insertion of interlocking screws through intramedullary nails/rods.

The various embodiments described above provide a drill and implant device and method for using the same. The device is configured to automatically drill a hole into an object, such as a bone, stop once a desired depth has been reached, and subsequently insert an implant into the hole. The device is able to perform the drilling and the implantation without applying a physical force to the device. Further, the user may leave the device in a stationary position with respect to the handle during the drilling and the implantation. The device is able to automatically drill into a bone to different depths depending on whether the device is in a manual mode, a first cortex drill mode, a second cortex find mode, a second cortex drill mode, a fixed depth mode, or a lag screw mode.

Although the various embodiments described above utilize the device for surgical applications, the device may be used for other applications as well. For example, the device may be used for construction applications, home improvement applications, and various other types of applications.

A medical device is summarized as including: a handle including a support portion; a housing coupled to the handle; a rail in the housing and on the support portion, the rail including a rail platform configured to move along the rail; a carriage in the housing and on the rail platform, the carriage including a threaded portion and a mounting portion; a lead screw in the housing and extending through the threaded portion; a linear motor in the housing and coupled to the lead screw, the linear motor configured to rotate the lead screw; a chuck in the housing and on the mounting portion, the chuck configured to hold a bit; a drill motor in the housing, on the mounting portion, and coupled to the chuck, the drill motor configured to rotate the chuck; and a nose cone coupled to the housing and aligned with the chuck and the drill motor.

The medical device further includes: a user interface on the housing, the user interface configured to display information to a user and receive input from the user.

The medical device further includes: a bearing structure coupled between a sidewall of the housing and a first end of the lead screw.

The medical device further includes: a shaft coupling coupled between a second end, opposite to the first end, of the lead screw and the linear motor.

The mounting portion includes a through hole, and the drill motor extends through the through hole.

The housing includes a door that exposes the chuck when the door is opened.

The medical device further includes: a linear controller configured to control the linear motor; a drill controller configured to control the drill motor; and an application controller configured to control the linear controller and the drill controller.

The application controller is configured to: control the linear controller to move, by the linear motor, the carriage to a calibration position; control the drill controller to rotate, by the drill motor, the chuck when the carriage is in the calibration position; and determine, concurrently with the chuck being rotated, a baseline torque value based on a torque of the linear motor or the drill motor.

The application controller is configured to: control the drill controller to rotate, by the drill motor, the chuck; control the linear controller to move, by the linear motor, the carriage concurrently with the chuck being rotated; and detect a first cortex based on a first current torque value of the linear motor and the baseline torque value.

The application controller is configured to: determine a first cortex baseline torque value based on the first current torque value; and determine a completion of a drilling of the first cortex based on a second current torque value of the linear motor and the first cortex baseline torque value.

The application controller is configured to: determine an intramedullary cavity baseline torque value based on the second current torque value; and detect a second cortex based on a third current torque value of the linear motor and the intramedullary cavity baseline torque value.

The application controller is configured to: determine a second cortex baseline torque value based on the third current torque value; and determine a completion of a drilling of the second cortex based on a fourth current torque value of the linear motor and the second cortex baseline torque value.

The linear motor includes a motor encoder configured to translate each determined rotation amount of the linear motor into an electrical pulse, and transmit the electrical pulse to the application controller, and the application controller is configured to: control the drill controller to rotate, by the drill motor, the chuck; control the linear controller to move, by the linear motor, the carriage concurrently with the chuck being rotated; detect a first cortex based on a first current torque value of the linear motor and the baseline torque value; start, in response to detection of the first cortex, a step counter; and increment the step counter in response to the electrical pulse being received from the motor encoder.

The application controller is configured to: determine a depth of the bit based on the step counter and a pitch or lead of the lead screw.

The medical device further includes: radiolucent attachment including: a secondary housing; a connector extending out of the secondary housing and configured to couple to the chuck; a plurality of gears in the secondary housing, the plurality of gears including a first gear and a second gear, the first gear coupled to the connector; and a secondary chuck or a bit coupled to the second gear.

The secondary housing and the plurality of gears are made of a radiolucent material.

A device is summarized as including: a handle; a housing coupled to the handle; a rail in the housing, the rail including a rail platform; a carriage in the housing and on the rail platform, the carriage including a threaded portion and a mounting portion; a lead screw in the housing and coupled to the threaded portion; a linear motor in the housing and coupled to the lead screw; a chuck in the housing and on the mounting portion; and a drill motor in the housing, on the mounting portion, and coupled to the chuck.

The linear motor includes a motor encoder configured to translate a determined rotation amount of the linear motor into an electrical pulse, and transmit the electrical pulse, and the device includes an application controller configured to: determine a baseline torque value based on a first torque value of the linear motor; rotate the chuck with the drill motor; move the carriage with the linear motor concurrently with the chuck being rotated; detect a first cortex based on a second torque value of the linear motor and the baseline torque value; start, in response to detection of the first cortex, a step counter; increment the step counter in response to the electrical pulse being received from the motor encoder; and determine a depth of a bit in the chuck based on the step counter and a pitch or lead of the lead screw.

A method is summarized as including: loading a bit into a device, the device including: a rail having a rail platform; a carriage on the rail platform, the carriage including a threaded portion and a mounting portion; a lead screw coupled to the threaded portion; a linear motor coupled to the lead screw; a chuck on the mounting portion, the bit being loaded into the chuck; and a drill motor on the mounting portion and coupled to the chuck; moving, by the linear motor, the carriage to a calibration position; rotating, by the drill motor, the chuck when the carriage is in the calibration position; and determining, concurrently with the chuck being rotated, a baseline torque value based on a torque of the linear motor or the drill motor.

The method further includes: rotating, by the drill motor, the chuck; moving, by the linear motor, the carriage concurrently with the chuck being rotated; and detecting a first cortex based on a first current torque value of the linear motor and the baseline torque value.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A medical device, comprising:
a handle including a support portion;
a housing coupled to the handle;
a rail in the housing and on the support portion, the rail including a rail platform configured to move along the rail;
a carriage in the housing and on the rail platform, the carriage including a threaded portion and a mounting portion;
a lead screw in the housing and extending through the threaded portion;
a linear motor in the housing and coupled to the lead screw, the linear motor configured to rotate the lead screw;
a chuck in the housing and on the mounting portion, the chuck configured to hold a bit;
a drill motor in the housing, on the mounting portion, and coupled to the chuck, the drill motor configured to rotate the chuck; and
a nose cone coupled to the housing and aligned with the chuck and the drill motor.

2. The medical device of claim 1, further comprising:
a user interface on the housing, the user interface configured to display information to a user and receive input from the user.

3. The medical device of claim 1, further comprising:
a bearing structure coupled between a sidewall of the housing and a first end of the lead screw.

4. The medical device of claim 3, further comprising:
a shaft coupling coupled between a second end, opposite to the first end, of the lead screw and the linear motor.

5. The medical device of claim 1 wherein the mounting portion includes a through hole, and the drill motor extends through the through hole.

6. The medical device of claim 1 wherein the housing includes a door that exposes the chuck when the door is opened.

7. The medical device of claim 1, further comprising:
a linear controller configured to control the linear motor;
a drill controller configured to control the drill motor; and
an application controller configured to control the linear controller and the drill controller.

8. The medical device of claim 7, wherein the application controller is configured to:
control the linear controller to move, by the linear motor, the carriage to a calibration position;
control the drill controller to rotate, by the drill motor, the chuck when the carriage is in the calibration position; and
determine, concurrently with the chuck being rotated, a baseline torque value based on a torque of the linear motor or the drill motor.

9. The medical device of claim 8, wherein the application controller is configured to:
control the drill controller to rotate, by the drill motor, the chuck;
control the linear controller to move, by the linear motor, the carriage concurrently with the chuck being rotated; and
detect a first cortex based on a first current torque value of the linear motor and the baseline torque value.

10. The medical device of claim 9, wherein the application controller is configured to:
determine a first cortex baseline torque value based on the first current torque value; and
determine a completion of a drilling of the first cortex based on a second current torque value of the linear motor and the first cortex baseline torque value.

11. The medical device of claim 10, wherein the application controller is configured to:
determine an intramedullary cavity baseline torque value based on the second current torque value; and
detect a second cortex based on a third current torque value of the linear motor and the intramedullary cavity baseline torque value.

12. The medical device of claim 11, wherein the application controller is configured to:
determine a second cortex baseline torque value based on the third current torque value; and
determine a completion of a drilling of the second cortex based on a fourth current torque value of the linear motor and the second cortex baseline torque value.

13. The medical device of claim 8, wherein
the linear motor includes a motor encoder configured to translate each determined rotation amount of the linear motor into an electrical pulse, and transmit the electrical pulse to the application controller, and
the application controller is configured to:
control the drill controller to rotate, by the drill motor, the chuck;
control the linear controller to move, by the linear motor, the carriage concurrently with the chuck being rotated;
detect a first cortex based on a first current torque value of the linear motor and the baseline torque value;
start, in response to detection of the first cortex, a step counter; and
increment the step counter in response to the electrical pulse being received from the motor encoder.

14. The medical device of claim 13, wherein the application controller is configured to:
determine a depth of the bit based on the step counter and a pitch or lead of the lead screw.

15. The medical device of claim 1, further comprising:
radiolucent attachment including:
a secondary housing;
a connector extending out of the secondary housing and configured to couple to the chuck;
a plurality of gears in the secondary housing, the plurality of gears including a first gear and a second gear, the first gear coupled to the connector; and
a secondary chuck or a bit coupled to the second gear.

16. The medical device of claim 15 wherein the secondary housing and the plurality of gears are made of a radiolucent material.

17. A device, comprising:
a handle;
a housing coupled to the handle;
a rail in the housing, the rail including a rail platform;
a carriage in the housing and on the rail platform, the carriage including a threaded portion and a mounting portion;
a lead screw in the housing and coupled to the threaded portion;
a linear motor in the housing and coupled to the lead screw;
a chuck in the housing and on the mounting portion; and
a drill motor in the housing, on the mounting portion, and coupled to the chuck.

18. The device of claim 17, wherein
the linear motor includes a motor encoder configured to translate a determined rotation amount of the linear motor into an electrical pulse, and transmit the electrical pulse, and
the device includes an application controller configured to:
determine a baseline torque value based on a first torque value of the linear motor;
rotate the chuck with the drill motor;
move the carriage with the linear motor concurrently with the chuck being rotated;
detect a first cortex based on a second torque value of the linear motor and the baseline torque value;
start, in response to detection of the first cortex, a step counter;
increment the step counter in response to the electrical pulse being received from the motor encoder; and
determine a depth of a bit in the chuck based on the step counter and a pitch or lead of the lead screw.

19. A method, comprising:
loading a bit into a device, the device including:
a rail having a rail platform;
a carriage on the rail platform, the carriage including a threaded portion and a mounting portion;
a lead screw coupled to the threaded portion;
a linear motor coupled to the lead screw;
a chuck on the mounting portion, the bit being loaded into the chuck; and
a drill motor on the mounting portion and coupled to the chuck;
moving, by the linear motor, the carriage to a calibration position;
rotating, by the drill motor, the chuck when the carriage is in the calibration position; and
determining, concurrently with the chuck being rotated, a baseline torque value based on a torque of the linear motor or the drill motor.

20. The method of claim 19, further comprising:
rotating, by the drill motor, the chuck;
moving, by the linear motor, the carriage concurrently with the chuck being rotated; and
detecting a first cortex based on a first current torque value of the linear motor and the baseline torque value.
